(19) United States
(12) Patent Application Publication
(10) Pub. No.: US 2021/0308318 A1
(43) Pub. Date: Oct. 7, 2021

(54) WOUND DRESSING SYSTEM

(71) Applicant: 3M INNOVATIVE PROPERTIES COMPANY, SAINT PAUL, MN (US)

(72) Inventors: DAVID R. HOLM, HUDSON, WI (US); ROBERT A. ASMUS, HUDSON, WI (US)

(73) Assignee: 3M INNOVATIVE PROPERTIES COMPANY, SAINT PAUL, MN (US)

(21) Appl. No.: 17/267,280

(22) PCT Filed: Aug. 14, 2019
(86) PCT No.: PCT/IB2019/056915

§ 371 Date: Feb. 9, 2021

Related U.S. Application Data
(60) Provisional application No. 62/765,015, filed on Aug. 17, 2018.

(51) Int. Cl. A61L 15/60; A61M 1/00; A61L 15/58; A61L 15/24; A61L 15/20

(52) U.S. Cl. CPC A61L 15/60; A61M 1/985; A61L 15/20; A61L 15/24; A61L 15/58

(57) ABSTRACT

A wound dressing system comprising a drape and a hydrogel sealant. The drape comprises a film and an adhesive on a first major surface of the film. The hydrogel comprises at least 30% by weight water, a crosslinked hydrophilic absorbent polymer; and a swelling agent. The hydrogel can create a seal between the drape and a surface (e.g., a patient's skin), making the disclosed systems suitable for negative pressure wound therapy (NPWT) applications.

### FIELD OF INVENTION

The present invention relates to wound dressing systems. The systems are particularly useful for applications in negative pressure wound therapy (NPWT).

### BACKGROUND

Wound dressings, and in particular film dressings, are widely used to protect a wound site from contaminating liquids and bacteria while also regulating moisture at the wound site to facilitate healing. Such dressings are available under a number of trade names such as TEGADERM™ (3M Company, St. Paul, Minn.), BIOCLUSIVE™ (Johnson & Johnson Company, New Brunswick, N.J.), and OP-SITE™ (T.J. Smith & Nephew, Hull, England).

Some wound dressings have been used in combination with negative pressure wound therapy (NPWT) to enhance the healing of particularly severe acute and chronic wounds. NPWT creates a vacuum or reduced pressure under the dressing which is used to draw fluids out of the wound and increase blood flow to the wound tissue. Such applications require a sufficient seal between the dressing and patient's skin so that a vacuum or reduced pressure environment can be maintained over the wound site for an extended period of time. The ability to maintain an effective seal can be frustrated by wounds on or around irregular surfaces, such as the knee, elbow, shoulders, heel, and ankle.

Despite current advances in wound dressings, there is still a need for wound dressings that are easy to use, conform well to rounded or irregularly shaped surfaces, provide a secure seal to maintain a sterile environment and, in the case of NPWT, maintain a vacuum or reduced pressure at the wound site over an extended period of time.

### SUMMARY

The present disclosure relates to wound dressing systems that can be used for a variety of applications, including NPWT. A common problem associated with NPWT is the inability to maintain a vacuum or reduced pressure over the wound site due to an inadequate seal between the dressing and patient's skin. Maintaining an effective seal can be particularly problematic when the dressing is placed on or in proximity to body parts that are highly contoured, frequently in motion, subject to high shear and/or exposed to excessive moisture (e.g., knee, foot, heel and sacrum). Inadequate seals reduce the healing effect of NPWT and increase caregiver time and expense to continually re-apply a vacuum or reduced pressure at the wound site. The present disclosure provides a wound dressing system that is conformable, easy to apply, and maintains a vacuum or reduced pressure at the would site for an extended period of time during NPWT.

In one embodiment, the present disclosure provides a wound dressing system comprising a drape and a hydrogel. The drape comprises a film and an adhesive on a first major surface of the film and has an inverted MVTR of at least 1,500 g/m² for 24 hours at 37° C. and 19% RH. The hydrogel comprises at least 30% by weight water, a crosslinked hydrophilic absorbent polymer, and a swelling agent.

In another embodiment, the present disclosure provides a method of applying the wound dressing system to a wound comprising the steps of applying the hydrogel to a patient's skin surrounding the perimeter of the wound, positioning the drape over the wound and hydrogel so that the first major surface of the film faces the wound, and coupling the drape to the patient's skin, wherein the drape and hydrogel form a sealed enclosure over the wound.

The above summary of the present disclosure is not intended to describe each disclosed embodiment or every implementation of the present disclosure. The description that follows more particularly exemplifies illustrative embodiments.

### BRIEF DESCRIPTION OF THE DRAWINGS

FIG. 1 is a schematic side view of one embodiment of a wound dressing system in the present disclosure;

FIG. 2 is a schematic perspective view of a NPWT set-up including a wound dressing system of the present disclosure;

FIG. 3 is a schematic cross-sectional view of the NPWT set-up in FIG. 2;

FIG. 4A is a schematic cross-sectional view of a test apparatus for monitoring the pressure in a vacuum seal;

FIG. 4B is a schematic top-view of a portion of the test apparatus in FIG. 4a; and

FIG. 5 is a schematic side view of the test apparatus in FIGS. 4a and 4b with a test sample.

Unless otherwise indicated, all figures and drawings in this document are not to scale and are chosen for the purpose of illustrating different embodiments of the invention. In particular, the dimensions of the various components are depicted in illustrative terms only, and no relationship between the dimensions of the various components should be inferred from the drawings, unless so indicated.

### DETAILED DESCRIPTION

Before any embodiments of the present disclosure are explained in detail, it is to be understood that the phraseology and terminology used herein is for the purpose of description and should not be regarded as limiting. As used herein, the terms “including,” “comprising,” or “having” and variations thereof encompass the items listed thereafter and equivalents thereof, as well as additional items. All numerical ranges are inclusive of their endpoints and non-integral values between the endpoints unless otherwise stated. Terms such as “top,” “bottom,” and the like are only used to describe elements as they relate to one another, but are in no way meant to recite specific orientations of an embodiment, to indicate or imply necessary or required orientations of the embodiment, or to specify how the embodiments described herein will be used, mounted, displayed, or positioned in use. As used herein, the term “overlie(s)” means to extend over so as to at least partially cover another layer or element. Overlying layers can be in direct or indirect contact (e.g., separated by one or more additional layers). As used herein, the term “coupled” and variations thereof is used broadly to encompass both direct and indirect couplings. The nature of such couplings can include chemical coupling means (e.g., via chemical bonds), mechanical coupling means, thermal coupling means; electrical coupling means, or combinations thereof.

The present disclosure generally relates to conformable wound dressing systems that create an effective seal between the dressing and a patient's skin, making the disclosed wound dressing systems ideally suited for NPWT applications. The systems comprise a high MVTR drape and a soft extrudable hydrogel as a sealant. The hydrogel is comprised of at least 30% by weight water, a crosslinked hydrophilic absorbent polymer, and a swelling agent. In practice, a bead of hydrogel is applied to a patient's skin surrounding the perimeter of the wound, and the drape is applied over the wound and hydrogel to form a sealed enclosure over the wound. Over time, water from the hydrogel evaporates through the drape, and the viscosity, cohesiveness and adhesiveness of the hydrogel increases, further insuring an effective seal between the drape and patient's skin. The swelling agent preferably does not plasticize the adhesive or the high MVTR film of the drape, which keeps the hydrogel stable and minimizes adhesive residue on the patient's skin during dressing changes. The drape adhesive preferably contains carboxylic acid groups that interact with the cross-linked absorbent polymer (e.g., carbonyl groups of polyvinyl pyrrolidone) of the hydrogel to further facilitate removal of the hydrogel during dressing changes.

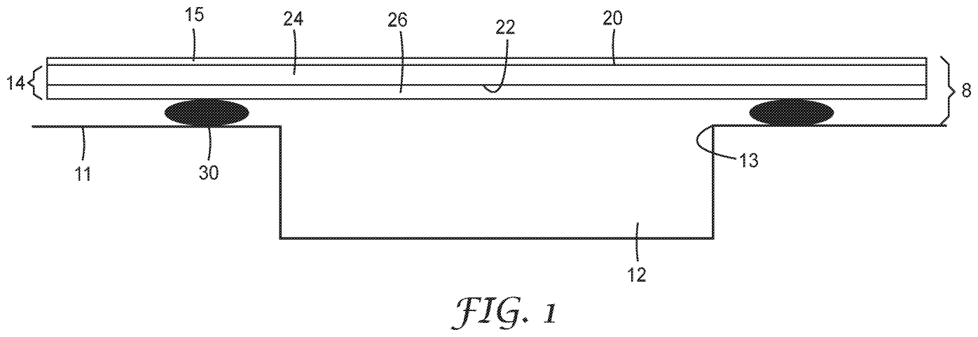

FIG. 1 illustrates one embodiment of a wound dressing system of the present disclosure. The wound dressing system 8 comprises a drape 14 and a hydrogel 30 that can function as a sealant between the drape 14 and a patient's skin 11. The drape 14 completely covers the wound 12 and secures to the skin 11 surrounding the wound perimeter 13.

The drape 14 comprises a film 24 and an adhesive 26 on a first major surface 22 of the film 24. The film 24 can be composed of a single layer of material or multiple layers of material and is typically conformable, liquid-impervious, and moisture vapor permeable. Suitable films have a composition and thickness that allow for the passage of moisture vapor through them. In some embodiments, the film is 15-40 μm thick, more particularly 20-30 μm thick. The film typically has an inverted MVTR of at least 2,500 g/m² for 24 hours at 37° C. and 19% RH, more particularly at least 10,000 g/m² for 24 hours at 37° C. and 19% RH using the inverted cup method for a 25 μm thick sample. The film typically has an inverted MVTR of no more than 40,000 g/m² for 24 hours at 37° C. and 19% RH using the inverted cup method for a 25 μm thick sample. In some embodiments, the film has an inverted MVTR of 2,500-40,000 g/m² for 24 hours at 37° C. and 19% RH, more particularly 10,000-40,000 g/m² for 24 hours at 37° C. and 19% RH using the inverted cup method for a 25 μm thick sample. In some embodiments, the film is transparent so that a caregiver can monitor the wound site without having to prematurely remove the dressing.

The adhesive 26 helps secure the drape 14 to the patient's skin 11 and/or other surface. Typically, the adhesive on the first major surface of the film is discontinuous in order to achieve a high moisture vapor transmission rate through the drape. The term “discontinuous” means that that the surface of the film on which the adhesive is applied has regions covered by adhesive (adhesive regions) and regions free of adhesive (adhesive-free regions). The adhesive-free regions can be random or patterned. In some embodiments, the adhesive regions cover at least 40%, more particularly at least 75% of the first major surface of the film. In some embodiments, the adhesive covers less than 95%, more particularly less than 85% of the first major surface of the film. In some embodiments, the adhesive covers 40-95%, more particularly 75-85% of the first major surface of the film. A discontinuous adhesive can be achieved, for example, by pattern coating, as described in U.S. Pat. Nos. 4,595,001 and 7,947,366. Alternatively, the discontinuous adhesive can be achieved by a converting step where an adhesive layer is perforated between two liners and the adhesive then laminated to a major surface of the film. The perforation can be accomplished with a laser or using mechanical means. In some embodiments, the adhesive dry coat weight is between 20 and 200 g/m².

Typically, the adhesive/film laminate (i.e., the drape) has an inverted MVTR of at least 1,500 g/m² for 24 hours at 37° C. and 19% RH, more particularly 5,000 g/m² for 24 hours at 37° C. and 19% RH, and even more particularly 10,000 g/m² for 24 hours at 37° C. and 19% RH using the inverted cup method for a 25 μm thick sample. Typically, the adhesive/film laminate has an inverted MVTR of no more than 20,000 g/m² for 24 hours at 37° C. and 19% RH using the inverted cup method for a 25 μm thick sample. In some embodiments, the inverted MVTR of the adhesive/film laminate is 1,500-20,000 g/m² for 24 hours at 37° C. and 19% RH, more particularly 5,000-20,000 g/m² for 24 hours at 37° C. and 19% RH, and even more particularly 10,000-20,000 g/m² for 24 hours at 37° C. and 19% RH using the inverted cup method for a 25 μm thick sample.

The wound dressing system 8 may optionally include a carrier 15 that overlies a second major surface 20 of the film 24. The carrier 15 is typically made of a material that is substantially more rigid than the film 24 and helps prevent the film 24 from wrinkling or folding back on itself during application of the dressing. The carrier can be made of one or more pieces that together form a layer that is co-extensive with one major surface of the film. Alternatively, the carrier can be made of one or more pieces that cover less than 100% of the area of one major surface of the film. The carrier 15 can be heat-sealable to the film, with or without low adhesion backsize (LAB) coating. The carrier 15 is generally removed from the film 24 after the drape 14 is secured to the patient's skin 11 and/or other surface.

The wound dressing system 8 may also optionally include a LAB coating (not shown) on the second major surface 20 of the film 24 such that the low adhesion coating is sandwiched between at least a portion of the carrier 15 and the second major surface 20 of the film 24. The low adhesion coating can cover the entire second major surface 20 of the film 24 or partially cover the second major surface 20 of the film 24 (e.g., discontinuous). A suitable low adhesion coating for use in the present dressings can be found, for example, in U.S. Pat. No. 5,531,855 (Example 1), which is compatible with a heat seal bond. The low adhesion coating reduces dressing changes due to unwanted dressing removal when other tapes or devices are placed on the dressing and subsequently removed, and reduces the surface friction of the dressing on linen or other fabrics, thereby offering additional protection against the accidental removal of the dressing. The low adhesion coating can also be used to facilitate removal of any carrier from the second major surface of the film.

The wound dressing system 8 optionally includes a release liner (not shown) that overlies and protects the adhesive 26 during storage and is removed right before application of the drape 14 to the patient. In some embodiments, the liner is a structured release liner, as further described below.

The hydrogel 30 provides a seal between the drape 14 and a patient's skin 11. The hydrogel typically comprises at least 30% by weight water, a cross-linked hydrophilic polymer, a swelling agent, and optional modifying polymer that increases cohesiveness while reducing adhesiveness. The hydrogel is generally of a sufficient viscosity that it can be delivered from a tube. The hydrogel is simple to apply and, unlike some previous sealants, does not need to undergo a curing process prior to use.

In practice, and with reference to FIG. 1, a caregiver would size the drape 14 so that is completely covers the wound 12 and the tissue proximate the wound perimeter 13. The edge or perimeter of the drape should be sufficient to cover the hydrogel or, as illustrated in FIG. 1, extend beyond the region of tissue to which the hydrogel is applied. A bead of hydrogel 30 is applied to the tissue proximate the wound perimeter 13 so that upon application of the wound dressing system, the hydrogel completely surrounds the wound. The optional release liner is removed from the adhesive 26 and the drape 14 is applied over the wound 12 and hydrogel 30 to form a sealed enclosure over the wound 12. The optional carrier 15 is then removed from the film 12.

Various features of the wound dressing system 8 are described in further detail below.

### Film

The drape comprises a liquid-impervious, moisture-vapor permeable polymeric film. The film can be composed of a single layer of material or multiple layers of material that may be coextruded together and/or bonded together with, for example, an adhesive

The film is preferably conformable to anatomical surfaces and can flex with movement of a joint, such as a knee. When a joint is flexed and then returned to its unflexed position, the film preferably stretches to accommodate the flexion of the joint but is resilient enough to continue to conform to the joint when the joint is returned to its unflexed condition. In some embodiments, the film has an ultimate elongation of greater than 200%, more particularly greater than 400%.

In some embodiments, the film is transparent to permit the user to monitor the wound through the drape.

The film preferably absorbs moisture and will increase in volume upon contact with moisture. The absorbent film facilitates the extraction of moisture from the hydrogel (described below) resulting in an increase in viscosity, cohesive strength and adhesive strength of the hydrogel. In some embodiments, the mass of the film increases at least 5%, more particularly at least 20%, and even more particularly at least 50% upon contact with water as determined according to the Water Absorption Test Method described in the Examples. In some embodiments, the film increases no more than 65% by weight upon contact with water. In some embodiments, the film increases 5-65% by weight upon contact with water.

The film can comprise one or more types of monomer (e.g., copolymers) and/or a mixture (e.g., blend) of polymers. Suitable films can be found, for example, in issued U.S. Pat. Nos. 5,088,483 and 5,160,315. In some preferred embodiments, the film is an elastomeric polyurethane, a co-polyester, a polyether block amide, and combinations thereof. These films combine the desirable properties of resiliency, high moisture vapor permeability, and transparency found in preferred films.

Suitable commercially available polymeric films include synthetic organic polymers including, but not limited to: polyurethanes commercially available from Lubrizol (Wickliffe, Ohio) under the trade designation ESTANE, including ESTANE® 58237 and ESTANE® 80MVT; polyether block amides commercially available from Arkema (King of Prussia, Pa.) under the trade designation Pebax, including Pebax® MV 3000, Pebax® MV1074 and Pebax® MH1657; poly-ester block copolymers commercially available from DuPont (Wilmington, Del.) under the trade designation HYTREL; elastomeric thermoplastic copolyesters available from DSM (Troy, Mich.) under the trade designation Arnitel, including Arnitel® VT3104 or Arnitel® VT3108.

### Adhesive

The adhesive is preferably a pressure sensitive adhesive. While any pressure sensitive adhesive can be used, it is preferable that the pressure sensitive adhesive be reasonably skin compatible and “hypoallergenic”. Suitable adhesives include acrylic, silicone and polyurethane adhesives. Exemplary adhesives include the silicone adhesives disclosed in PCT Publication Nos. WO 2010/056541 and WO 2010/056543 and the silicone adhesive elastomers disclosed in U.S. Pat. No. 6,497,724. Other suitable pressure sensitive adhesives are described in U.S. Pat. Nos. 3,389,827; 4,112,213; 4,310,509; and 4,323,557.

Preferably, the pressure sensitive adhesive contains acid groups that interact with C═O groups of the cross-linked absorbent polymer in the hydrogel (described below). The interaction facilitates removal of the hydrogel with the drape when removing or changing the wound dressing system. In some embodiments, the acid group is a carboxylic acid group and, in more particular embodiments, an acrylic acid. In some preferred embodiments, the adhesive can comprise 2-20% by weight acrylic acid. If the percentage of acrylic acid is greater than 20%, the adhesive tends to become stiff and does not stick well to skin. If the percentage of acrylic acid is below 2%, the adhesive cannot sufficiently bind to the hydrogel, thus increasing the chance that hydrogel residue will be left on a surface (e.g., patient's skin) when the wound dressing system is removed. Suitable pressure sensitive adhesives having acid groups include acrylic copolymers described in U.S. Pat. No. RE 24,906, a 97:3 iso-octyl acrylate:acrylamide copolymer, and a 70:15:15 isooctyl acrylate:ethyleneoxide acrylate:acrylic acid terpolymer, as described in U.S. Pat. No. 4,737,410 (Example 31).

In some embodiments, the pressure sensitive adhesive is an acrylic copolymer or a silicone gel, more preferably an acrylic copolymer.

Inclusion of antimicrobial agents (described further below) and/or other therapeutic agents in the adhesive is also contemplated, an example of which is described in U.S. Pat. Nos. 4,310,509 and 4,323,557.

### Release Liner

Release liners can be made, for example, of kraft papers, polyethylene, polypropylene, polyester or composites of any of these materials. The liners are preferably coated with release agents such as fluorochemicals or silicones. In some preferred embodiments, the liners are papers, polyolefin films, or polyester films coated with silicone release materials. Examples of commercially available release liners include POLYSLIK™ silicone release papers available from Loparex (Cary, N.C.), Silicone 1750 coated films from Infiana (Forchheim, Germany), siliconized polyethylene terephthalate films available from H.P. Smith Co. (Stoneham, Mass.), and 3M Scotchpak™ 9741 Release liner from 3M Company (St. Paul, Minn.).

In some preferred embodiments, a structured liner having raised features is used to imprint a reverse image on the exposed surface of the adhesive. The height of the raised features, as determine from the base of the liner, will depend to some extent on the thickness of the adhesive. In some embodiments, the height of the raised features is equal to at least the thickness of the adhesive, where the thickness refers to the largest value as measured from the surface of the film for a continuous or discontinuous adhesive. In preferred embodiments, at least some of the raised features will intersect, thus creating channels in the adhesive layer upon removal of the liner. The channels provide a mechanism for distributing moisture throughout the adhesive layer which can facilitate transmission of moisture through the drape. The channels also provide a greater surface area over which the hydrogel can bond to the adhesive, facilitating removal of the hydrogel from a surface (e.g., skin) upon removal or changing the wound dressing system. Over time, the adhesive can migrate to fill in the channels left by the structured liner, increasing adhesion of the film to the surface. Applicants have also discovered that structured release liners can prevent wrinkling of the drape during sterilization with ethylene oxide. Commercially available structured liners include polyethylene or polypropylene liners from Infiana (Forchheim, Germany) with embossing structure D124 (embossing height 350 μm), D119 (embossing height 150 μm), and the A4 design (embossing height 110 μm).

Other combinations of adhesives and liners are contemplated for use with embodiments according to the present invention. Those skilled in the art will be familiar with the processes of testing a new adhesive against different liners or a new liner against different adhesives to arrive at the combination of qualities desired in a final product. The considerations pertinent to the selection of a silicone release liner can be found, for example, in Chapter 18 of the Handbook of Pressure Sensitive Adhesive Technology, Van Nostrand-Reinhold, 1982, pp. 384-403. U.S. Pat. No. 4,472,480 also describes considerations pertinent to the selection of a perfluoropolyether release liner.

### Carrier (Optional)

The carrier is made of a material that is substantially more rigid than the film to prevent the film from wrinkling or folding back on itself during application. The carrier can be heat-sealable to the film, with or without the low adhesion coating. In general, the carrier comprises at least one of a polyethylene/vinyl acetate copolymer, a polyvinylacetate coated paper, a polyester film, or a polyethylene film. The carrier can also include other materials such as nonwovens, additional polymer films, or papers attached to at least a portion of the aforementioned polymer films.

### Hydrogel

The hydrogel sealant comprises at least 30% by weight water, a crosslinked hydrophilic absorbent polymer, a swelling agent, and an optional modifying polymer. Each of the components is described further below.

### Crosslinked Hydrophilic Absorbent Polymer

In most embodiments, the crosslinked hydrophilic absorbent polymer is poly(N-vinyl lactam). Poly(N-vinyl lactam) polymers can be provided in any form susceptible to crosslinking, such as the solid forms described in U.S. Pat. Nos. 4,931,282; 5,225,473; and 5,389,376. Nonlimiting examples of solid forms include particles, pellets, sheets, flakes, and bulk objects of various shapes, and coated objects of various shapes. Typically, the poly(N-vinyl lactam) is in the form of particles of a size less than about 1 cm in diameter, more typically from about 0.1 micron to 0.250 cm and often from about 10 microns to about 1000 microns. Alternatively, the poly(N-vinyl) lactam can be crosslinked in solution.

Poly(N-vinyl lactam) can be a noncrosslinked homopolymer or a noncrosslinked copolymer containing N-vinyl lactam monomeric units, which after crosslinking becomes swellable in a swelling agent and is biocompatible with mammalian (e.g., human) skin. Nonlimiting examples of N-vinyl lactam monomers are N-vinyl-2-pyrrolidone; N-vinyl-2-valerolactam; N-vinyl-2-caprolactam; and mixtures of any of the foregoing. Preferably, the N-vinyl lactam is N-vinyl-2-pyrrolidone. Nonlimiting examples of comonomers useful with the aforementioned N-vinyl lactam monomers include N,N-dimethylacrylamide, acrylic acid, methacrylic acid, hydroxyethylmethacrylate, acrylamide, 2-acrylamido-2-methyl-1-propane sulfonic acid or its salt, and vinyl acetate. Normally, N-vinyl lactam monomeric units will comprise no less than about 50 weight percent of the monomeric units present in the poly(N-vinyl lactam) in solid state form. Typically, N-vinyl lactam monomeric units comprise a majority of total monomeric units of the polymer, and more typically, the N-vinyl lactam monomeric units comprise 70 to 100 percent by weight of the poly(N-vinyl lactam) and often 90 to 100 percent by weight of the poly(N-vinyl lactam). Typically, the poly(N-vinyl lactam) is a homopolymer of N-vinyl-2-pyrrolidone.

Noncrosslinked N-vinyl lactam homopolymer and N-vinyl pyrrolidone/vinyl acetate copolymers are commercially available. Nonlimiting sources of commercially available poly(N-vinyl pyrrolidone) useful for the present invention include Aldrich Chemical Co. (Milwaukee, Wis.), BASF (Parsippany, N.J.), Ashland (Covington, Ky.), Dan River Corporation (Danville, Va.), and Spectrum Chemical Manufacturing Corporation (Gardena, Calif.). Poly(N-vinyl lactam) can have a Fikentscher K-value of at least K-15, and normally at least K-60 more often K-90, or even K-120. Other Fikentscher K-values are possible. Fikentscher K-values are described in Molyneaux, Water-Soluble Polymers: Properties and Behavior, Vol. 1, CRC Press, 1983, pp. 151-152.

The poly(N-vinyl lactam) can be radiation-crosslinked while the lactam is in a solid form. After exposure to ionizing radiation, poly(N-vinyl lactam) can have a Swelling Capacity in water of at least about 15, typically at least about 30, and often at least about 40 as described in U.S. Pat. No. 5,409,966 and summarized in the Examples.

Alternatively, poly (N-vinyl lactam) can be crosslinked by free-radical polymerization, either in bulk or in solution, of a precursor containing an N-vinyl lactam monomer, optionally other monomers, and a crosslinking compound as described in U.S. Pat. No. 4,931,282.

The amount of swellable crosslinked poly(N-vinyl lactam) in the hydrogel is typically from about 3 to about 20 weight percent, exclusive of any biocompatible and/or therapeutic materials to be added to the hydrogel. When the poly(N-vinyl lactam) is poly(N-vinyl pyrrolidone), the weight percent is preferably from about 4 to about 15 percent, more particularly from about 5 to 10 percent.

In addition to the poly (N-vinyl lactam) polymers mentioned above, the crosslinked hydrophilic absorbent polymer may also include cross-linked polyacrylic acid (PAA) (e.g., CARBOPOL™ No. 974P; B.F. Goodrich, Brecksville, Ohio) and copolymers of such, polyacrylamides, and combinations thereof.

### Swelling Agent

The hydrogel also comprises a swelling agent which can swell both the crosslinked poly(N-vinyl lactam) polymer and the modifying polymer, and which is biocompatible with human skin. The swelling agent typically has an average molecular weight of 75-2,500 g/mole.

Suitable swelling agents include polyglycerols, such as glycerol, diglycerol, polyglycerol-3, tetraglycerol, hexaglycerol and decaglycerol. Preferably, the average molecular weight of the polyglycerol is between 180 and 750 g/mol.

The swelling agent can be added in an amount ranging from about 15 to about 70 weight percent of the hydrogel and preferably from about 20 to about 50 weight percent, exclusive of any biocompatible and/or therapeutic materials to be added to the hydrogel. In some embodiments, the swelling agent is polyglycerol-3. Polyglycerol-3 can comprise up to 100 weight percent of the swelling agent. The ratio (w/w) of swelling agent to total absorbent polymer (e.g., poly(N-vinyl lactam) and optional modifying polymer) should be at least 2.5, and preferably at least 3.4. In some embodiments, at least 71% by weight of the dry gel is the swelling agent, exclusive of any biocompatible and/or therapeutic materials.

### Modifying Polymer

The modifying polymer is present in the hydrogel composition to maintain and/or increase cohesiveness while reducing adhesiveness. When added with the swelling agent, the modifying polymer becomes solubilized or suspended in the swelling agent. Typically, the modifying polymer will form a viscous solution or viscous gel when combined with the swelling agent in a weight ratio of modifying polymer to swelling agent of 1:8 to 1:10.

The choice of swelling agent typically will determine the appropriate modifying polymer to accomplish a reduction in adhesion while maintaining or improving cohesion of the adhesive composition. Modifying polymers that are poorly solubilized in one swelling agent may be highly swollen in a different swelling agent for use in the present invention. In some embodiments, examples of suitable modifying swellable polymers include, but are not limited to, polysaccharides, polysaccharide derivatives, acrylates, acrylate derivates, cellulose, cellulose derivatives, and combinations thereof.

In particular embodiments, modifying swellable polymers for use in the present invention are hydroxypropyl guar; guar gum; hydroxyethyl cellulose; hydroxypropyl cellulose; hydroxypropyl methylcellulose; polymeric quaternary ammonium salt of hydroxyethyl cellulose reacted with trialkyl ammonium substituted epoxide; copolymers of hydroxyethyl cellulose and diallyldimethyl ammonium chloride; and derivatives and combinations of the foregoing.

The amount of modifying polymer can range up to about 10 weight percent of the hydrogel, exclusive of any biocompatible and/or therapeutic materials. Consequently, the weight percent of the modifying polymer can be from about 0.1 to about 8 percent. When the modifying polymer is hydroxypropyl guar, the weight percent of hydroxypropyl guar can range from about 1 to about 5 percent.

### Water

Water makes up 30-85% by weight of the hydrogel, preferably 45-75% by weight of the hydrogel. The water levels and swelling agent levels should be sufficient to aid in squeezing or extruding the hydrogel from a plastic or foil tube or packet and spreading onto the skin prior to the drape be applied to the body part. Upon evaporation of a majority of the water during wear, the viscosity, cohesiveness, and adhesiveness of the hydrogel will rapidly increase to ensure minimal movement of the hydrogel during wear which provides high confidence in the continued vacuum seal performance of the hydrogel. Yet, the hydrogel remains sufficiently soft to maintain an excellent vacuum seal by readily filling in cracks and crevices in the skin, and possible folds in the drape that occur during application. The hydrogel is also skin friendly and is easy to clean up with water or sterile saline.

In some embodiments, 5-10% by weight of the water can be replaced with lower molecular weight monohydric alcohols (e.g., ethanol and isopropanol) and/or miscible or highly water-soluble diols (e.g., ethylene glycol, propylene glycol, PEG 400, PEG 600). The alcohols and diols can decrease the dry time of the hydrogel and enhance antimicrobial performance. However, they can also pose problems to those with skin sensitivity. Therefore, typically no more than 10% of the water is replace with the lower molecular weight monohydric alcohols and/or miscible or highly water-soluble diols.

### Buffer (Optional)

Hydrogels with strongly acidic or basic pH values can be buffered to provide a more skin friendly experience. Typically, the hydrogels are buffered within a dermatologically acceptable pH range of 4-8. Exemplary buffering agents include citric acid/sodium citrate dihydrate buffer solution and acetic acid/sodium acetate buffer solution.

### Antimicrobial Agents

The adhesive and/or hydrogel can optionally contain one or more antimicrobials to reduce the likelihood of an infection or to treat infections of the skin or wound.

There are numerous biologically active materials, which include antimicrobial agents. Examples of antimicrobial agents include Parachlorometaxylenol; triclosan; Chlorhexidine and its salts such as Chlorhexidine Gluconate, Octenidine and its salts, poly hexamethylene biguanide and its salts such as poly hexamethylene biguanidine chloride, iodine, iodophors; fatty acid monoesters; poly-n-vinyl pyrrolidone-iodophors; silver oxide, silver and its salts, peroxides (e.g. hydrogen peroxide), antibiotics (e.g. neomycin, bacitracin, and polymixin B).

The following active ingredients could also be used to suppress the regrowth or possibly treat an infection of microorganisms: 2,2-thiobis(4-chlorophenol); 4,4-isopropylidenediphenol; 5-amino-6-chloro-o-cresol; acetaminosalol; alcloxa; aldioxa; aluminum acetate; aluminum benzoate; aluminum diacetate; aluminum formate; aluminum phenolsulfonate; ammonium iodide; ammonium phenolsulfonate; benzisothiazolinone; benzotriazole; benzoxiquine; benzylparaben; berberine chloride; boric acid; cetethyl morpholinium ethosulfate; cetethyldimonium bromide; cetrimonium tosylate; cetylpyridinium chloride; chloramine-t; chlorothymol; cloflucarban; cocotrimonium chloride; colloidal sulfur; copper usnate; dedm hydantoin; dedm hydantoin dilaurate; dequalinium acetate; dequalinium chloride; dibromopropamidine diisethionate; dichloro-m-xylenol; dichlorophene; dichlorophenyl imidazoldioxolan; diiodomethyltolylsulfone; dimethyl hydroxymethylpyrazole; dimethylaminostyryl heptyl methyl thiazolium iodide; dodecylbenzyltrimonium chloride; domiphen bromide; ferulic acid; fluorosalan; glyoxal; hydroxymethyl dioxoazabicyclooctane; hydroxypropyl bistrimonium diiodide; ichthammol; isodecylparaben; isopropyl sorbate; lapyrium chloride; laurtrimonium trichlorophenoxide; lauryl isoquinolinium bromide; lauryl isoquinolinium saccharinate; laurylpyridinium chloride; m-cresol; mandelic acid; MDM hydantoin; MEAa-iodine; melaleuca alternifolia; methylbenzethonium chloride; mixed cresols; nonoxynol-12 iodine; nonoxynol-9 iodine; o-cresol; oxyquinoline benzoate; oxyquinoline sulfate; p-chlorophenol; p-cresol; PEG-15 dedm hydantoin; PEG-15 dedm hydantoin stearate; PEG-5 dedm hydantoin; PEG-5 dedm hydantoin oleate; phenol; phenoxyethylparaben; phenyl salicylate; polymethoxy bicyclic oxazolidine; potassium iodide; potassium lactate; potassium phenoxide; potassium troclosene; quaternium-14; quaternium-24; quaternium-8; ricinoleamidopropyltrimonium methosulfate; sodium iodide; sodium p-chloro-m-cresol; sodium phenolsulfonate; sodium phenoxide; sodium usnate; steapyrium chloride; strontium peroxide; tea-sorbate; tetrabutyl ammonium bromide; thiabendazole; triacetin; undecylenamide dea; undecylenamide mea; undecylenamidopropyltrimonium methosulfate; undecyleneth-6; undecylenoyl peg-5 paraben; usnic acid; zinc acetate; zinc borate; zinc phenolsulfonate; zinc sulfate; zinc undecylenate; and combinations of the foregoing.

The following actives could also be of use to also reduce regrowth of microorganisms on skin: 2-bromo-2-nitropropane-1,3-diol; 4-hydroxybenzoic acid; 5-bromo-5-nitro-1,3-dioxane; 7-ethylbicyclooxazolidine; ammonium benzoate; ammonium bisulfate; ammonium propionate; ammonium sulfite; behentrimonium chloride; benzalkonium bromide; benzalkonium chloride; benzalkonium saccharinate; benzethonium chloride; benzoic acid; benzyl alcohol; benzylhemiformal; bromochlorophene; butyl benzoate; butylparaben; calcium benzoate; calcium paraben; calcium propionate; calcium salicylate; calcium sorbate; calcium undecylenate; cetalkonium chloride; cetearalkonium bromide; cetrimonium bromide; cetrimonium chloride; chloroacetamide; chlorobutanol; chlorophene; chloroxylenol; chlorphenesin; climbazole; dehydroacetic acid; diazolidinyl urea; dibromohexamidine isethionate; dichlorobenzyl alcohol; dimethyl oxazolidine; DMDM hydantoin; ethyl benzoate; ethylparaben; formaldehyde; formic acid; glutaral; hexamidine; hexamidine diisethionate; hexamidine paraben; hexetidine; hydrogenated tallowtrimonium chloride; imidazolidinyl urea; iodopropynyl butylcarbamate; isobutyl benzoate; isobutylparaben; isopropyl benzoate; isopropyl cresols; isopropylparaben; lauralkonium bromide; lauralkonium chloride; laurtrimonium bromide; laurtrimonium chloride; magnesium benzoate; magnesium propionate; magnesium salicylate; MEA o-phenylphenate; MEA-benzoate; MEA-salicylate; MEA-undecylenate; methenamine; methyl benzoate; methylchloroisothiazolinone; methyldibromo glutaronitrile; methylisothiazolinone; methylparaben; myristalkonium chloride; myristalkonium saccharinate; myrtrimonium bromide; o-cymen-5-ol; o-phenylphenol; olealkonium chloride; p-chloro-m-cresol; phenoxyethanol; phenoxyisopropanol; phenyl benzoate; phenyl mercuric acetate; phenyl mercuric benzoate; phenyl mercuric borate; phenyl mercuric bromide; phenyl mercuric chloride; phenylparaben; piroctone olamine; polyaminopropyl biguanide; potassium benzoate; potassium butylparaben; potassium ethylparaben; potassium metabisulfite; potassium methylparaben; potassium o-phenylphenate; potassium paraben; potassium propionate; potassium propylparaben; potassium salicylate; potassium sorbate; potassium sulfite; propionic acid; propyl benzoate; propylparaben; quaternium-15; salicylic acid; sodium benzoate; sodium bisulfate; sodium butylparaben; sodium dehydroacetate; sodium ethylparaben; sodium formate; sodium hydroxymethylglycinate; sodium iodate; sodium metabisulfite; sodium methylparaben; sodium o-phenylphenate; sodium paraben; sodium propionate; sodium propylparaben; sodium salicylate; sodium sorbate; sodium sulfite; sodium undecylenate; sorbic acid; soytrimonium chloride; stearalkonium chloride; steartrimonium chloride; tallowalkonium chloride; tallowtrimonium chloride; thimerosal; triclocarban; triclosan; undecylenic acid; zinc pyrithione; and combinations of the foregoing.

### NPWT Applications

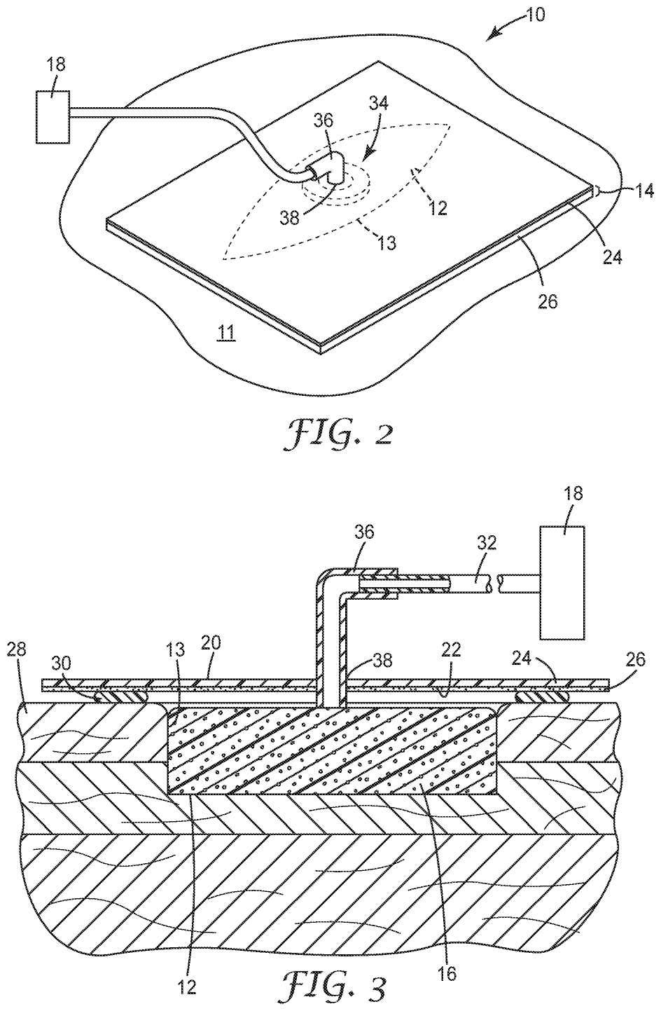

FIGS. 2 and 3 illustrate one embodiment of a NPWT set-up 10 including a wound dressing system of the present disclosure. Like reference numerals used to describe the wound dressing system in FIGS. 1-3 refer to like components. A drape 14 comprising a film 24 and adhesive 26 is sized to cover the wound and a portion of the skin surrounding the wound perimeter 13. The adhesive 26 can be used to couple the drape to the patient's epidermis 28 or another layer, such as a gasket or additional sealing member.

A bead of hydrogel 30 extends around the wound perimeter 13 and further couples the drape 14 to the patient's epidermis. The hydrogel fluidly (e.g., hermetically) seals the drape 14 against the patient's epidermis 28. As used herein, a fluid seal refers to a seal which maintains a vacuum or reduced pressure over the wound for a period of time after the vacuum source is removed. In some embodiments, a reduced pressure is maintained under the drape for at least 1 hour, more preferably at least 10 hours, and even more preferably at least 20 hours based upon the Vacuum Seal test method disclosed below. In some embodiments, the pressure under the drape changes by no more than 15% within 2 minutes, preferably 5 hours, even more preferably 20 hours after removal of the vacuum source using the Vacuum Seal test method disclosed below.

A manifold 16 is disposed proximate or within the wound 12. The term “manifold” as used herein generally refers to a substance or structure that is provided to assist in applying negative or reduced pressure to, delivering fluid to, or removing fluids from a tissue site or wound 12.

Examples of manifolds 16 may include, for example, but are not limited to, devices that have structural elements arranged to form flow channels, such as, for example, cellular foam, open-cell foam, porous tissue collections, nonwoven materials, woven materials, liquids, gels, foams that include, or cure to include, flow channels, or combinations thereof. The manifold 16 may be porous and may be made from foam, nonwoven materials, woven materials, felted mat, or any other material suited to a particular biological application. In some embodiments, the manifold 16 can be a porous foam and include a plurality of interconnected cells or pores that act as flow channels. The porous foam may be a polyurethane, open-cell, reticulated foam, such as V.A.C.® GranuFoam® material manufactured by Kinetic Concepts, Incorporated of San Antonio, Tex. Other embodiments may include closed-cell foams. In some situations, the manifold 16 may also be used to distribute fluids such as medications, antibacterials, growth factors, and various solutions to the wound 12. Other layers may be included in or on the manifold 16, such as absorptive materials, wicking materials, hydrophobic materials, and hydrophilic materials.

With continued reference to FIGS. 2 and 3, the reduced pressure supplied by the negative or reduced-pressure source 18 (e.g., pump) can be delivered through a conduit 32 to a reduced-pressure interface 34, which, in some embodiments, can include an elbow port 36. The reduced-pressure interface 34, e.g., a connector, can be disposed proximate the manifold 16 and can extend through an aperture 38 in the drape 14. In some embodiments, the port 36 can be a TRAC® technology port available from Kinetic Concepts, Inc. of San Antonio, Tex. The reduced-pressure interface 34 allows the reduced pressure to be delivered to the wound 12. In this illustrative embodiment, the port 36 extends through the drape 14 to the manifold 16.

### EXAMPLES

Materials

| Item | Description | Source |
|---|---|---|
| Citric Acid | | VWR International (West |
| | | Chester, PA) |
| Sodium citrate | | VWR International (West |
| | | Chester, PA) |
| Acetic acid | | VWR International (West |
| | | Chester, PA) |
| Sodium acetate | | VWR International (West |
| | | Chester, PA) |
| Estane ® 58237 | Polyurethane resin | Lubrizol (Wickliffe, OH) |
| Polypropylene | | Felix Schoeller Group |
| coated paper | | (Osnabrück, Germany) |
| Polyglycerol-3 | | Solvay (Princeton, NJ) |
| ECOPOL-120 | Hydroxy propyl | Economy Polymers & |
| | guar | Chemicals (Houston, TX) |
| PHMB | Polyhexanide | Arch Chemicals, Inc. |
| | (polyhexamethylene | (Alpharetta, GA) |
| | biguanide) | |
| PVP K-90D | Poly(N-vinyl | Ashland Chemical |
| | pyrrolidone) | (Columbus, OH) |
| Silicone coated | | Loparex (Cary, NC) |
| paper release liner | | |
| REF M8275052/5 | Negative Pressure | Acelity (San Antonio, TX) |
| V.A.C. | Wound Therapy Kit | |
| GRANUFOAM | | |
| Dressing | | |
| 50 micron polyester | | Loparex (Cary, NC) |
| film | | |
| Sterile Water | | Baxter Healthcare |
| | | (Deerfield, IL) |
| VITRO-SKIN ® | Testing substrate that | IMS Inc. (Portland, ME) |
| | claims to effectively | |
| | mimic the surface | |
| | properties of human | |
| | skin | |
| DT-8890 Digital | Digital Differential | CEM (Kolkata, India) |
| Manometer | Air Vapor Pressure | |
| | Meter Gauge | |
| | Manometer | |

### Test Methods

### Viscosity

Performed with an Ares G2 Rheometer from TA Instruments (New Castle, Del.) using a 40 mm diameter APS heat break, stainless steel, parallel plate with a gap of 1 mm at 25° C. Equilibration time was 120 second. Pre-shear shear rate was 0.16/s for 120 second. The oscillation-frequency strain was 1.58%. The angular frequency was 0.1 to 100 rad/s (five points per decade).

### Water Absorption

Water absorption was measured according to ASTM D570. Samples were approximately 50.8 mm in diameter and approximately 3.2 mm thick. The samples were immersed in distilled water at 23° C. for 24 hours.

### Inverted Moisture Vapor Transmission Rate (MVTR)

The MVTR of dressings in contact with liquid water were measured according to EN 13726-2:2002, Section 3.3 using 19% Relative Humidity and 37° C. in the chamber.

### Swelling Capacity

Swelling Capacity, S, is a measurement of the milliliters of water sorbed per gram of solid, radiation-crosslinked poly(N-vinyl lactam), approximated by equation I:

$$S = \frac{[ ( PVP WT . + {H}_{2} O WT . ) \times \frac{GEL HEIGHT}{TOTAL HEIGHT} ] - PVP WT .}{PVP WT .} \qquad I$$

where PVP wt. is the poly(N-vinyl lactam) weight, H₂O WT is water weight, Gel Height is height of swollen poly(N-vinyl lactam) in a vial, and Total Height is total height of material in the vial.

Swelling Capacity correlates to a measurement of polymer swelling as a function of chemical crosslinking units in poly(N-vinyl lactam), according to the equation II:

S=C(λ^{1/3}−λₒ^{1/3}) II

where S is a measurement of water sorbed per gram of poly(N-vinyl lactam), C is a constant characteristic of the poly(N-vinyl lactam), i.e., the volume of water sorbed per gram of poly(N-vinyl lactam), λ is the average number of backbone carbon atoms in the polymer segments between crosslinked junctions, and λ₀ is the average number of backbone carbon atoms between crosslinked junctions when S is zero. Swelling capacity and this equation are discussed in Errede, “Molecular Interpretations of Sorption in Polymers Part I”, Advances in Polymer Science Vol. 99, Springer-Verlag, Berlin Heidelberg Germany (pp. 21-36, 1991).
pH

The pH of the sample was then measure with a Symphony B30PCI pH Instrument (VWR International).

### Vacuum Seal

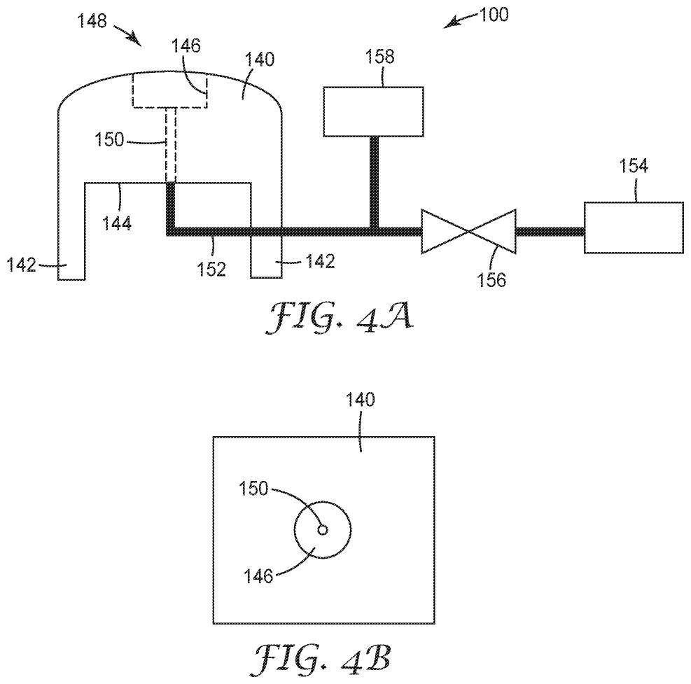

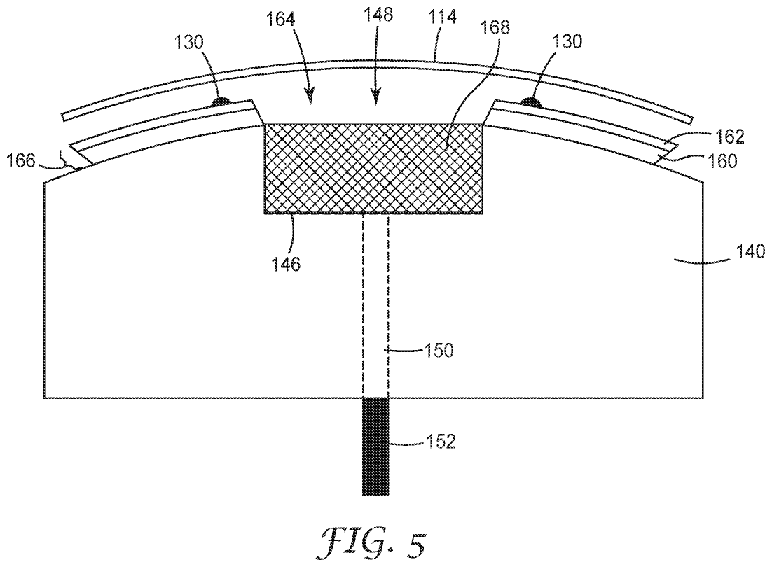

As illustrated in FIGS. 4A, 4B and 5, a test system 100 was developed to monitor vacuum loss from a wound dressing applied to simulated skin with a simulated wound. A quarter segment of a sphere (i.e., sphere segment) 140 was machined out of aluminum and mounted on four legs 142 secured to the flat side 144 of the sphere segment 140. The sphere segment 140 had a diameter of 25.4 cm and each of the legs 142 had a diameter of 2.54 cm. A 5 cm diameter well 146 having a depth of 1.8 cm was machined at the apex (or pole) 148 of the sphere segment 140. A 6 mm bore hole 150 extended from the bottom of the well 146 to the side of the sphere opposite the apex. One end of a pipe fitting (not shown) was secured in the bore hole 150 and the other end of the pipe fitting was attached to a vacuum line 152 connected to a vacuum pump 154. A ball valve 156 was placed in the vacuum line 152 between the bore hole fitting and the vacuum pump 154. A DT-8890 digital manometer 158 was placed between the bore hole fitting and the ball valve 156 to monitor the pressure in the test system 100.

An approximately 1 mm thick sheet (20 cm×20 cm) of hydrophilic adhesive gel 160 made as described in U.S. Pat. No. 9,278,155 (Example 73) was laminated to a similar size sheet of VITRO-SKIN® (IMS Inc., Portland, Me.) 162 and allowed to equilibrate for 24 hours at 23° C. and 50% relative humidity. A 4.75 cm diameter hole 164 was punched out of the center of the laminate 166, and the laminate 166 was placed on top of the sphere segment 140 such that the center of the hole 164 in the gel/VITRO-SKIN® laminate 166 lined up with the center of the well 146 at the apex 148 of the sphere segment 140.

Two pieces of foam 168 from negative wound pressure kit REF M8275052/5 V.A.C. GRANUFOAM Dressing Medium Featuring Sensa T.R.A.C. Technology were cut into 5 cm diameter circles and placed in the well 146 at the apex 148 of the sphere segment 140.

A 15 cm×15 cm transparent drape 114 was placed over the foam 168 and onto the gel/VITRO-SKIN® 162 such that the drape 114 created a border of approximately 5 cm or more around the foam 168. In examples where hydrogel of the present invention was used, a bead of the hydrogel 130 was applied around the circumference of the wound prior to applying the drape 114 to the gel/VITRO-SKIN® 162. A vacuum of 200 mm Hg below atmospheric pressure was created with the vacuum pump 154. The ball valve 156 was then closed and the vacuum below atmospheric pressure was monitored over time.

### Example 1 (Film)

A 23 micron thick film of Estane® 58237 polyurethane resin was extruded onto a polypropylene coated paper. The inverted MVTR of the film was 23,000 g/m² for 24 hours at 37° C. and 19% RH as measured according to EN 13726-2:2002, Section 3.3 using 19% Relative Humidity in the chamber.

### Example 2 (Adhesive)

A 70:15:15 isooctyl acrylate:ethyleneoxide acrylate:acrylic acid terpolymer adhesive was solution polymerized as described in U.S. Pat. No. 4,737,410 (Example 31).

### Example 3 (Hydrogel)

A uniform gel was prepared by mixing the following ingredients in a high shear mixer: 23.85 grams of polyglycerol-3; 67.86 grams of distilled water; 0.97 grams of hydroxyl propyl guar (ECOPOL-120); and 7.32 grams of poly(N-vinyl pyrrolidone) (PVP K-90D) that had been gamma irradiated at approximately 110 kGy.

### Example 4 (Hydrogel)

A uniform gel was prepared by mixing the following ingredients in a high shear mixer: 42.5 grams of polyglycerol-3; 50.0 grams of distilled water; and 7.5 grams of gamma irradiated poly(N-vinyl pyrrolidone).

### Example 5 (Hydrogel)

A uniform gel was prepared by mixing the following ingredients in a high shear mixer: 33.9 grams of polyglycerol-3; 57.5 grams of distilled water; and 8.5 grams of gamma irradiated poly(N-vinyl pyrrolidone).

### Example 6 (Adhesive/Film Laminate)

The adhesive from Example 2 was applied in a discontinuous pattern (23% open, 2.3 mm holes in a regular pattern, 50 g/m² coat weight) to one side of the film from Example 1. The water in contact moisture vapor transmission rate of the adhesive/film laminate was measured to be 3960±270 g/m² for 24 hours at 37° C. and 19% RH.

### Example 7 (Comparative Adhesive/Film Laminate)

The adhesive from Example 2 was flood coated on a silicone coated paper release liner at a dry coat weight of 50 g/m². The adhesive was then laminated to the film from Example 1 and the release liner removed. The water in contact moisture vapor transmission rate of the adhesive/film laminate was measured to be 860±30 g/m² for 24 hours at 37° C. and 19% RH.

### Example 8 (Comparative Adhesive/Film Laminate)

A transparent film drape dressing from the negative pressure wound therapy kit REF M8275052/5 V.A.C GRANUFOAM Dressing Medium Featuring Sensa T.R.A.C Technology was purchased from EBay. The water in contact moisture vapor transmission rate was measured to be 400±10 g/m² for 24 hours at 37° C. and 19% RH.

### Examples 9-10 Loss of Moisture from Gel

A specified mass of gel from Example 3 and Example 4 was weighed onto a 2 mil thick polyester film (12.5 cm×12.5 cm) and covered with the adhesive/film laminate (10 cm×10 cm) from Example 6. The mass of the sample was weighed periodically to determine the loss in mass of the gel (presumable moisture) at 23° C. and 50% relative humidity. The results are shown in Table 1 below.

TABLE 1
Moisture Loss

| Time | Example 3 | Example 4 |
|---|---|---|
| (Hour) | Mass (g) of Gel from | Mass (g) of Gel from |
| 0 | 0.51 | 0.48 |
| 1 | 0.39 | 0.43 |
| 2 | 0.34 | 0.39 |
| 3 | 0.29 | 0.36 |
| 4 | 0.27 | 0.34 |
| 5 | 0.25 | 0.32 |
| 6 | 0.23 | 0.30 |

### Examples 11-12 (Moisture Loss)

A specified mass of gel from Example 5 was extruded via a tube in the shape of a circle (about 38 mm in diameter) onto a 50 micron thick polyester film (approximately 20 cm×20 cm) from Loparex (Cary, N.C.), and then covered by the drapes described in Example 6 or Example 8 (Comparative). The drape sizes were approximately 15 cm×15 cm. The mass of the gel/film/drape system was recorded over time at 23° C. and approximately 50% relative humidity. Assuming any loss in mass was attributed to water in the gel, the total mass of the gel overtime was calculated. The results are shown in Table 2 below.

TABLE 2
Moisture Loss

| | Example 11 Using | | Example 12 (Comparative) |
|---|---|---|---|
| Time | Drape from Example 6 | Time | Using Drape from Example |
| (hour) | Mass (g) of Gel | (hour) | 8 Mass (g) of Gel |
| 0 | 1.57 | 0 | 1.50 |
| 0.8 | 1.47 | 0.9 | 1.49 |
| 3.6 | 1.21 | 3.7 | 1.45 |
| 4.7 | 1.15 | 4.8 | 1.45 |
| 6.2 | 1.08 | 6.3 | 1.42 |
| 23.4 | 0.74 | 23.5 | 1.18 |

### Examples 13-16 (Hydrogel Viscosity)

Hydrogels were prepared by mixing various amount of distilled water with 4 parts polyglycerol-3, and 1 part gamma irradiated poly (N-vinyl pyrrolidone). The viscosities are reported in Table 3.

TABLE 3
Viscosities

| | | Viscosity (Pa · S) |
|---|---|---|
| Example | % (w/w) Water in Gel | at 0.25 rad/sec |
| 13 | 70 | 240 |
| 14 | 60 | 330 |
| 15 | 55 | 430 |
| 16 | 50 | 910 |

### Example 17 (Vacuum Seal Test)

The gel from Example 15 was squeezed out of a polyethylene tube into a bead onto the VITRO-SKIN® around the simulated wound (about 10 mm from the simulated wound) of the vacuum seal test apparatus. A 15 cm×15 cm size dressing from Example 6 was then placed onto the vacuum seal test apparatus such that the drape extended at least 25 mm beyond the gel bead. The vacuum test was then conducted and the results are shown in Table 4 below.

TABLE 4
Vacuum Test

| | Vacuum (mm Hg below |
|---|---|
| Time (Minutes) | Atmospheric Pressure) |
| 0 | 205 |
| 2 | 203 |
| 9 | 197 |
| 30 | 191 |
| 104 | 184 |
| 218 | 181 |
| 1317 | 165 |

### Example 18 (Comparative Vacuum Seal Test)

A 15 cm×15 cm size dressing from Example 8 was placed onto the vacuum seal test apparatus and the pressure in the system was monitored over time. The results are shown in Table 5 below.

TABLE 5
Vacuum Test

| | Vacuum (mm Hg below |
|---|---|
| Time (Minutes) | Atmospheric Pressure) |
| 0 | 202 |
| 2 | 67 |
| 4 | 35 |
| 6 | 26 |
| 10 | 16 |
| 25 | 0 |

### Example 19

A 0.1 M citrate buffer solution of pH 5.5 was made by dissolving 5.80 grams of citric acid and 20.52 grams of sodium citrate dihydrate (VWR International, West Chester, Pa.) in a 1 L solution of sterile water (Baxter Healthcare, Deerfield, Ill.).

### Example 20

A 0.1 M acetate buffer solution of pH 5.5 was made by dissolving 0.62 grams of acetic acid and 7.35 grams of anhydrous sodium acetate (VWR International, West Chester, Pa.) in a 1 L solution of sterile water (Baxter Healthcare, Deerfield, Ill.).

### Example 21

A 0.2 weight percent solution of polyhexanide (PHMB) in sterile water was made by diluting 5 grams of a 20 weight percent solution of PHMB (Arch Chemicals, Inc. (Alpharetta, Ga.)) with 495 grams of sterile water.

### Example 22

A uniform gel was prepared by mixing the following ingredients in a high shear mixer: 28 grams of polyglycerol-3; 65 grams of sterile water; and 7 grams of gamma irradiated poly(N-vinyl pyrrolidone). The gel was enclosed in a high density polyethylene (HDPE) plastic bottle and then gamma irradiated at 29 kGy. The viscosity of the gel was determined 26 days after gamma irradiation. Results are shown in Table 6.

### Example 23

A uniform gel was prepared according to the procedure in Example 22, except that the acetate buffer solution from Example 20 was used instead of sterile water. The results after gamma irradiation are shown in Table 6.

### Example 24

A uniform gel was prepared according to the procedure in Example 22, except that the citrate buffer solution from Example 19 was used instead of sterile water. The results after gamma irradiation are shown in Table 6.

### Example 25

A uniform gel was prepared according to the procedure in Example 22, except that the PHMB solution from Example 21 was used instead of sterile water. The results after gamma irradiation are shown in Table 6.

TABLE 6
Viscosity of gels

| | Viscosity 26 days after gamma |
|---|---|
| Sample | irradiation (Pa · S at 0.25 rad/sec) |
| Example 22 | 325 |
| Example 23 | 256 |
| Example 24 | 253 |
| Example 25 | 323 |

1-20. (canceled)

21. A wound dressing system comprising:
a drape comprising a film and an adhesive on a first major surface of the film, the drape having an inverted MVTR of at least 1,500 g/m² for 24 hours at 37° C. and 19% RH; and
a hydrogel comprising at least 30% by weight water, a crosslinked hydrophilic absorbent polymer, and a swelling agent;
wherein the hydrogel is adjacent the drape first major surface.

22. The wound dressing system of claim 21, wherein the film absorbs 5 to 65% by weight water according to the Water Absorption Test Method.

23. The wound dressing system of claim 21, wherein the adhesive comprises acid groups, more particularly carboxylic acid groups.

24. The wound dressing system of claim 21, wherein the adhesive comprises 2-20% by weight acrylic acid.

25. The wound dressing system of claim 21, wherein the hydrogel comprises 3-20% by weight cross-linked hydrophilic absorbent polymer.

26. The wound dressing system of claim 21, wherein the cross-linked hydrophilic absorbent polymer comprises at least one of poly (N-vinyl lactam), poly acrylamides, and co-polymers thereof.

27. The wound dressing system of claim 21, wherein the cross-linked hydrophilic absorbent polymer comprises poly (N-vinyl pyrrolidone).

28. The wound dressing system of claim 27, wherein the cross-linked hydrophilic absorbent polymer has a swelling capacity of at least 15.

29. The wound dressing system of claim 21, wherein the hydrogel comprises 4-15% by weight of poly (N-vinyl pyrrolidone), exclusive of any biocompatible and/or therapeutic materials.

30. The wound dressing system of claim 21, wherein the swelling agent has a molecular weight of 75-2,500 g/mol.

31. The wound dressing system of claim 21, wherein the swelling agent comprises polyglycerols.

32. The wound dressing system of claim 31, wherein the polyglycerol has an average molecular weight of 180-750 g/mol.

33. The wound dressing system of claim 21, wherein the hydrogel further comprises a modifying polymer.

34. The wound dressing system of claim 33, wherein the ratio (w/w) of modifying polymer to swelling agent is 1:8 to 1:10.

35. The wound dressing system of claim 21, wherein the hydrogel continuously surrounds an area on the first major surface of the film.

36. The wound dressing system of claim 35, wherein the hydrogel is applied to a surface.

37. The wound dressing system of claim 36, wherein a reduced pressure can be maintained with the area over the surface.

38. The wound dressing system of claim 37, comprising an aperture in the drape within the area, wherein a negative pressure source connects to the aperture.
